(19) United States
(12) Patent Application Publication
(10) Pub. No.: US 2012/0202263 A1
(43) Pub. Date: Aug. 9, 2012

(54) Bioactive Macromers and Hydrogels and Methods for Producing Same

(75) Inventors: Brandon L. Blakely, Phoenix, AZ (US); Jordan Miller, Philadelphia, PA (US); Christopher S. Chen, Princeton, NJ (US)

(73) Assignee: THE TRUSTEES OF THE UNIVERSITY OF PENNSYLVANIA, Philadelphia, PA (US)

(21) Appl. No.: 13/365,326

(22) Filed: Feb. 3, 2012

Related U.S. Application Data
(60) Provisional application No. 61/439,006, filed on Feb. 3, 2011.

(51) Int. Cl. C12N 9/96; C07K 14/475; C12N 5/00; C07K 2/00; C07K 7/08

(52) U.S. Cl. 435/188; 530/411; 525/54.1; 530/327; 435/396; 530/399

(57) ABSTRACT

The invention concerns macromers, having a molecular weight of at least 2 kDa, comprising at least one unit of the formula P-(protein-P)ₙ, wherein: P is selected from polyethylene glycol (PEG), alginate, polyurethane, and polyvinyl alcohol; protein comprises at least one bis-cysteine matrix metalloproteinase (MMP)-sensitive peptide; and n is an integer from 2 to 500. Other aspects of the invention concern hydrogels utilizing cross-linked macromers and methods of producing such macromers and hydrogels.

### CROSS-REFERENCE TO RELATED APPLICATIONS

This patent application claims the benefit of U.S. Provisional Patent Application Ser. No. 61/439,006, “Bioactive Macromers And Hydrogels And Methods For Producing Same” filed Feb. 3, 2011, the entirety of which is incorporated by reference herein.

### STATEMENT OF GOVERNMENT SUPPORT

The research carried out in this application was supported, in part, by grants from the National Institute of Health (National Cancer Institute) through grant numbers EB00262, HL73305, and GM74048. Pursuant to 35 U.S.C. §202, the government may have rights in any patent issuing from this application.

### TECHNICAL FIELD

The present invention is directed to bioactive macromers and hydrogels and methods of making same.

### BACKGROUND

In the past several decades, engineered materials have become an increasingly important and versatile tool for mimicking the native in vivo environment, and provide unparalleled control over the cellular microenvironment compared to the substantially more complex naturally-derived materials [Lutolf M P, Hubbell J A. Synthetic biomaterials as instructive extracellular microenvironments for morphogenesis in tissue engineering. Nat. Biotechnol. 2005; 23:47-55]. Hydrogels, owing to their hydrophilic nature and ability to absorb large amounts of water, are one class of materials that have received significant attention for cell biology and tissue engineering applications [Tibbitt M W, Anseth K S. Hydrogels as extracellular matrix mimics for 3D cell culture. Biotechnol Bioeng. 2009; 103:655-63]. A widely investigated class of synthetic hydrogels is based on poly(ethylene glycol) (PEG), whose neutral charge, hydrophilicity, and resistance to protein adsorption make them biocompatible for both in vitro synthetic chemistry [Hill-West J L, Chowdhury S M, Sawhney A S, Pathak C P, Dunn R C, Hubbell J A. Prevention of postoperative adhesions in the rat by in situ photopolymerization of bioresorbable hydrogel barriers. Obstet. Gynecol. 1994; 83:59-64; West J, Hubbell J. Polymeric biomaterials with degradation sites for proteases involved in cell migration. Macromolecules. 1999; 32:241-4; Lutolf M P, Hubbell J A. Synthesis and physicochemical characterization of end-linked poly(ethylene glycol)-co-peptide hydrogels formed by Michael-type addition. Biomacromolecules. 2003; 4:713-22; Lutolf M P, Lauer-Fields J L, Schmoekel H G, Metters A T, Weber F E, Fields G B, et al. Synthetic matrix metalloproteinase-sensitive hydrogels for the conduction of tissue regeneration: engineering cell-invasion characteristics. Proc Natl Acad Sci USA. 2003; 100:5413-8; Seliktar D, Zisch A H, Lutolf M P, Wrana J L, Hubbell J A. MMP-2 sensitive, VEGF-bearing bioactive hydrogels for promotion of vascular healing. J Biomed Mater Res A. 2004; 68:704-16; Dikovsky D, Bianco-Peled H, Seliktar D. The effect of structural alterations of PEG-fibrinogen hydrogel scaffolds on 3-D cellular morphology and cellular migration. Biomaterials. 2006; 27:1496-506; Benoit D, Schwartz M, Durney A, Anseth K. Small functional groups for controlled differentiation of hydrogel-encapsulated human mesenchymal stem cells. Nat. Mater. 2008; 7:816-23; and Fairbanks B, Scott T, Kloxin C, Anseth K, Bowman C. Thiol—Yne Photopolymerizations: Novel Mechanism, Kinetics, and Step-Growth Formation of Highly Cross-Linked Networks. Macromolecules. 2009; 42:211-7]. While PEG alone is unable to support cellular activity, copolymers of PEG and biologically active moieties including peptides have been successfully applied in a diverse range of in vitro and in vivo studies. From a design perspective, the peptides or proteins conjugated to PEG are the main controls used to engineer the bioactive and bioresponsive character of these synthetic gels. PEG-peptide hydrogels have been utilized in the three-dimensional study of ensemble fibroblast migration [Gobin A S, West J L. Cell migration through defined, synthetic ECM analogs. FASEB J. 2002; 16:751-3; Lee S-H, Moon J J, Miller J S, West J L. Poly(ethylene glycol) hydrogels conjugated with a collagenase-sensitive fluorogenic substrate to visualize collagenase activity during three-dimensional cell migration. Biomaterials. 2007; 28:3163-70; and Raeber G P, Lutolf M P, Hubbell J A. Mechanisms of 3-D migration and matrix remodeling of fibroblasts within artificial ECMs. Acta Biomater. 2007; 3:615-29], chondrocyte maintenance for cartilage engineering [Elisseeff J, Anseth K, Sims D, McIntosh W, Randolph M, Yaremchuk M, et al. Transdermal photopolymerization of poly(ethylene oxide)-based injectable hydrogels for tissue-engineered cartilage. Plast Reconstr Surg. 1999; 104:1014-22 and Lee H J, Lee J-S, Chansakul T, Yu C, Elisseeff J H, Yu S M. Collagen mimetic peptide-conjugated photopolymerizable PEG hydrogel. Biomaterials. 2006; 27:5268-76], hepatocyte metabolism [Liu Tsang V, Chen A A, Cho L M, Jadin K D, Sah R L, DeLong S, et al. Fabrication of 3D hepatic tissues by additive photopatterning of cellular hydrogels. FASEB J. 2007; 21:790-801], valvular interstitial cell matrix secretion [Shah D N, Recktenwall-Work S M, Anseth K S. The effect of bioactive hydrogels on the secretion of extracellular matrix molecules by valvular interstitial cells. Biomaterials. 2008; 29:2060-72], and a range of other applications [Lutolf M P, Weber F E, Schmoekel H G, Schense J C, Kohler T, Müller R, et al. Repair of bone defects using synthetic mimetics of collagenous extracellular matrices. Nat. Biotechnol. 2003; 21:513-8; Mapili G, Lu Y, Chen S, Roy K. Laser-layered microfabrication of spatially patterned functionalized tissue-engineering scaffolds. J Biomed Mater Res B Appl Biomater. 2005; 75:414-24; and Hahn M S, McHale M K, Wang E, Schmedlen R H, West J L. Physiologic pulsatile flow bioreactor conditioning of poly(ethylene glycol)-based tissue engineered vascular grafts. Ann Biomed Eng. 2007; 35:190-200].

A variety of coupling chemistries and hydrogel architectures have been used, ultimately imparting PEG hydrogels with similar properties that are attractive in these diverse biomedical applications. West and Hubbell developed early hydrogels sensitive to the activity of matrix metalloproteinases (MMPs) made of block copolymers of degradable peptides and PEG, flanked with photopolymerizable acrylates [West, et al, Macromolecules. 1999; 32:241-4]. Later innovations by West and colleagues led to hydrogel redesign by reacting heterobifunctional acrylate-PEG-N-hydroxysuccinimide active esters with bis-amine MMP-sensitive peptides to form precursors of the form acrylate-PEG-peptide-PEG-acrylate [Gobin and West, FASEB J. 2002; 16:751-3 and Mann B K, Gobin A S, Tsai A T, Schmedlen R H, West J L. Smooth muscle cell growth in photopolymerized hydrogels with cell adhesive and proteolytically degradable domains: synthetic ECM analogs for tissue engineering. Biomaterials. 2001; 22:3045-51]. Hubbell and colleagues also introduced an approach using Michael-type addition between bis-cysteine MMP-sensitive peptides and 4-arm PEG-vinylsulfones to cross-link reactants into a hydrogel in a single step [Lutolf and Hubbell, Biomacromolecules. 2003; 4:713-22 and Seliktar, et al, J Biomed Mater Res A. 2004; 68:704-16]. Similarly, Anseth and colleagues have utilized multi-arm PEGs in thiol-ene photopolymerization [Aimetti A A, Machen A J, Anseth K S. Poly(ethylene glycol) hydrogels formed by thiol-ene photopolymerization for enzyme-responsive protein delivery. Biomaterials. 2009; 30:6048-54] and novel click-chemistries [DeForest C A, Polizzotti B D, Anseth K S. Sequential click reactions for synthesizing and patterning three-dimensional cell microenvironments. Nat. Mater. 2009; 8:659-64] to tailor the cellular microenvironment.

These bioactive PEG-based hydrogels are being explored as a scaffolding to support tissue engineering. Because these materials ultimately will be implanted in vivo to support thick multicellular constructs, the ability of such hydrogels to support angiogenesis—the physiologic sprouting of new blood vessels from existing ones—and vascular integration of an implant also will need to be optimized. Although angiogenesis has been extensively studied in natural materials such as collagen and fibrin gels [Krishnan L, Underwood C J, Maas S, Ellis B J, Kode T C, Hoying J B, et al. Effect of mechanical boundary conditions on orientation of angiogenic microvessels. Cardiovasc Res. 2008; 78:324-32 and Staton C A, Reed M W R, Brown N J. A critical analysis of current in vitro and in vivo angiogenesis assays. Int J Exp Pathol. 2009; 90:195-221], or Matrigel [Mammoto A, Connor K M, Mammoto T, Yung C W, Huh D, Aderman C M, et al. A mechanosensitive transcriptional mechanism that controls angiogenesis. Nature. 2009; 457:1103-8], investigators are only just beginning to examine how to engineer PEG-based hydrogels to support vascular ingrowth. Recent studies have shown promise via the encapsulation or immobilization of vascular endothelial growth factor (VEGF) [Zisch A H, Lutolf M P, Ehrbar M, Raeber G P, Rizzi S C, Davies N, et al. Cell-demanded release of VEGF from synthetic, biointeractive cell ingrowth matrices for vascularized tissue growth. FASEB J. 2003; 17:2260-2; Ehrbar M, Rizzi S C, Hlushchuk R, Djonov V, Zisch A H, Hubbell J A, et al. Enzymatic formation of modular cell-instructive fibrin analogs for tissue engineering. Biomaterials. 2007; 28:3856-66; and Leslie-Barbick J E, Moon J J, West J L. Covalently-immobilized vascular endothelial growth factor promotes endothelial cell tubulogenesis in poly(ethylene glycol) diacrylate hydrogels. J Biomater Sci Polym Ed. 2009; 20:1763-79] or Ephrin-A1 [Moon J J, Lee S-H, West J L. Synthetic biomimetic hydrogels incorporated with ephrin-A1 for therapeutic angiogenesis. Biomacromolecules. 2007; 8:42-9] in these materials.

PEG based hydrogels are commonly used for tissue engineered substrates as they are extremely biocompatible and are easy to be tuned to match the necessary mechanics of the replicated tissue. This particular construct is an improvement over other hydrogels as it is constructed from low weight starting material into large molecular weight macromers. Current hydrogels are made of elements from 2-20 kDa as this is the size that is clearable by the liver and kidneys. However, these are too small to produce a large enough mesh size to produce adequate angiogenesis, fibroblast migration, or neuronal spreading.

### SUMMARY

The present invention is directed to bioactive maromers and hydrogels and methods of making same. Certain aspects of the invention concern macromers comprising polymers and protein units.

In some embodiments, the invention concerns, synthetic hydrogels based on poly(ethylene glycol) (PEG) have been used as biomaterials for cell biology and tissue engineering investigations. Bioactive PEG-based gels have largely relied on heterobifunctional or multi-arm PEG precursors that can be difficult to synthesize and characterize or expensive to obtain. The present invention concerns an alternative strategy, which instead uses inexpensive and readily available PEG precursors to simplify reactant sourcing. This new approach provides a robust system in which to probe cellular interactions with the microenvironment. We used the step-growth polymerization of PEG diacrylate (PEGDA, 3400 Da) with bis-cysteine matrix metalloproteinase (MMP)-sensitive peptides via Michael-type addition to form biodegradable photoactive macromers of the form acrylate-PEG-(peptide-PEG)m-acrylate. The molecular weight (MW) of these macromers is controlled by the stoichiometry of the reaction, with a high proportion of resultant macromer species greater than 500 kDa. In addition, the polydispersity of these materials was nearly identical for three different MMP-sensitive peptide sequences subjected to the same reaction conditions. When photopolymerized into hydrogels, these high MW materials exhibit increased swelling and sensitivity to collagenase-mediated degradation as compared to previously published PEG hydrogel systems. Cell-adhesive acrylate-PEG-CGRGDS was synthesized similarly and its immobilization and stability in solid hydrogels was characterized with a modified Lowry assay. To illustrate the functional utility of this approach in a biological setting, we applied this system to develop materials that promote angiogenesis in an ex vivo aortic arch explant assay. We demonstrate the formation and invasion of new sprouts mediated by endothelial cells into the hydrogels from embedded embryonic chick aortic arches. Furthermore, we show that this capillary sprouting and three-dimensional migration of endothelial cells can be tuned by engineering the MMP susceptibility of the hydrogels and the presence of functional immobilized adhesive ligands (CGRGDS vs. CGRGES peptide). The facile chemistry described and significant cellular responses observed suggest the usefulness of these materials in a variety of in vitro and ex vivo biologic investigations, and may aid in the design or refinement of material systems for a range of tissue engineering approaches.

In some aspects, the invention concerns macromers, having a molecular weight of at least 2 kDa, comprising at least one unit of the formula

P-(protein-P)ₙ

wherein: P is selected from polyethylene glycol (PEG), alginate, polyurethane, and polyvinyl alcohol; protein comprises at least one bis-cysteine matrix metalloproteinase (MMP)-sensitive peptide or bis-amine protein; and n is an integer from 2 to 500. In some embodiments, P is PEG and n is an integer that is in the range of 50 to 150. In certain embodiments, the PEG has a molecular weight of about 2,000 to 40,000 Da. Some preferred PEGs are substantially linear.

Certain proteins contain or consist of a bis-cysteine matrix metalloproteinase (MMP)-sensitive peptide. In some embodiments, preferred proteins have at least one peptide having the sequence CGPQGIAGQGCR, CGPQGPAGQGCR or CGPQGIWGQGCR. In some compositions, the protein additionally comprises non-MMP-sensitive peptides. Certain proteins comprise an enzyme. Some proteins comprise a biologic growth factor.

In one aspect of the invention, a macromer of the invention is associated with at least one additional macromer of the invention such that the macromers are associated via one or more of cross-linking, hydrogen bonding, ionic, or van der Waals interactions.

In some embodiments, P comprises one or more of alginate, polyurethane, and polyvinyl alcohol

Another aspect of the invention concerns hydrogel tissue engineering scaffolds comprising a hydrogel derived from cross-linking of a macromer described herein.

Yet another aspect of the invention concerns methods of producing a bioactive hydrogel comprising:

step-growth polymerization of (i) protein comprising one or more bis-cysteine matrix metalloproteinase (MMP)-sensitive peptides and (ii) at least one of polyethylene glycol-divinylsulfone, polyethylene glycol-diacrylate, polyethylene glycol-diacrylamide or polyethylene glycol-dicarboxylic acid or derivatives thereof to produce macromers of the formula X-PEG-(peptide-PEG)n-X, at least 50% of said macromers having a molecular weight of at least 2 kDa; and

cross-linking said macromers to form the bioactive hydrogel;

wherein X is carboxylic acid, vinylsulfone, acrylate or acrylamide, PEG is polyethylene glycol, and n is 2 to 500.

Some methods utilize step-growth polymerization which is accomplished by Michael-type addition in aqueous solution having a basic pH. Other methods utilize an organic solvent. Certain methods conduct the step-growth polymerization step with a molar excess of (i) the moles of polyethylene glycol-diacrylate or polyethylene glycol-diacrylamide relative to (ii) the moles of bis-cysteine matrix metalloproteinase (MMP)-sensitive peptide.

In some embodiments, the cross-linking is accomplished by radical mediated photopolymerization. In other embodiments, the cross-linking is accomplished by hydrogen bonding or ionic interactions between said protein segments.

Certain methods produce macromers having a molecular weight of at least 500 kDa.

One preferred PEG-dicarboxylic acid or derivatives thereof, is PEG-di-N-hydroxysuccinimide or PEG-di-succinimidylcarboxymethylester and, in some embodiments, the protein is a bis-amine peptide or protein. In some embodiments, a mixture of acrylate-PEG-N-hydroxysuccinimide or acrylamide-PEG-N-hydroxysuccinimide and PEG-di-N-hydroxysuccinimide is utilized.

In certain embodiments, the step-growth polymerization to form the macromer is accomplished with ‘living’ polymerization methods between polyethylene glycol-diacrylate and polyethylene glycol-diacrylamide chains and bis-acrylate flanked amino acid sequences previously listed including metal ion catalyzed anionic and cationic polymerization (Aoshima, S., and Kanaoka, S. (2008) in Wax Crystal Control•Nanocomposites Stimuli-Responsive Polymers (Springer Berlin/Heidelberg), pp. 169-208). Is some embodiments the step-growth polymerization to form the macromer is accomplished with ‘living’ radical polymerization methods including reversible addition-fragmentation chain transfer (RAFT) using reversible transfer agents (Ganachaud, F., Monteiro, M. J., Gilbert, R. G., Dourges, M.-A., Thang, S. H., and Rizzardo, E. (2000). Molecular Weight Characterization of Poly(N-isopropylacrylamide) Prepared by Living Free-Radical Polymerization. Macromolecules 33, 6738-6745.) and transition metal catalyzed atom transfer radical polymerization (ATRP) (Masci, G., Giacomelli, L. and Crescenzi, V. (2004), Atom Transfer Radical Polymerization of N-Isopropylacrylamide. Macromolecular Rapid Communications, 25: 559-564. doi:10.1002/marc.200300140).

In some methods, the step-growth polymerization is controlled and defined a priori with block copolymer arrangements as dictated by order of reagent addition in polymerization. In certain methods, the step-growth polymerization is controlled to narrow polydispersity (<1.2).

For some methods, the step-growth polymerization to form the macromer is accomplished with radical thiol-ene ‘click’ reaction with appropriate radical imitator (Hoyle, C. and Bowman, C. (2010), Thiol-Ene Click Chemistry. Angewandte Chemie International Edition, 49: 1540-1573. doi: 10.1002/anie.200903924). In yet other methods, the step-growth polymerization can be designed to occur between multifunctional monomers capable of generating thiol-acrylate reactions, as previously described, in addition to orthogonal functionalities present on the monomers (such as alkyne and azide groups) for further functionalization using additional ‘click’ chemistries (such as Azide-Alkyne Huisgen Cycloaddition).

### BRIEF DESCRIPTION OF THE DRAWINGS

FIG. 1 presents (a) Synthetic scheme. PEG 3400 is reacted with acryloyl chloride to form PEGDA, which is then reacted with cysteine-bearing peptides via Michael-type addition to form cell adhesive or, in a separate reaction, MMP-sensitive PEG-acrylate macromers. Reaction stoichiometry controls the molecular weight and polydispersity of the resultant species during step-growth polymerization. (b) Schematic illustration of hydrogel structure. Photopolymerization of the photoactive precursors from (a) yields bioactive hydrogels with multiple MMP-sensitive peptides per backbone chain, with pendant cell-adhesive ligands tethered from sites of acrylate crosslinking.

FIG. 2 shows GPC analysis of MMP-sensitive PEG-diacrylates plotted against PEG MW standards. Highly degradable (“HD”) peptide reacted with a 2.2 molar excess of PEGDA (uppermost curve) via step-growth polymerization resulted in more than 80% conjugation (sum of medium and high MWs). Reaction of MMP-sensitive peptides with a 1.6 molar excess of PEGDA (remaining three curves) resulted in more than 90% conjugation, with a majority of the molecular weight species greater than 500 kDa. When subjected to the same reaction stoichiometry, HD, collagen native (“CN”), and least degradable (“LD”) PEG-peptide conjugates show nearly identical polydispersity.

FIG. 3 presents (a) MMP-sensitive hydrogels (made from HD, CN, or LD peptides) were polymerized at 10% w/w and then swollen to equilibrium over 36 h (n=3, ‘Eq. Swollen’ in figure). Bars indicate standard deviation. (b) Swollen hydrogels were degraded in 0.2 mg/mL collagenase (n=3) or incubated in buffer (n=1) up to 8 h while their wet weight was monitored. Note that HD and CN have overlapping degradation curves. Bars indicate standard deviation.

FIG. 4 shows the immobilization efficiency and stability of acrylate-PEG-peptide macromers in PEGDA gels was assessed with a modified Lowry assay for total protein concentration, as well as HUVEC seeding. (a) The Lowry assay, typically only used for large proteins, produced a linear standard curve from the short, soluble CGREDV peptide, even at low concentrations. (b) This standard curve was used to quantify the solution-based concentration of acrylate-PEG-CGRGDS and acrylate-PEG-CGRGES macromers, with a deviation from expected of 40-50%, with values comparable between both peptides. Bars indicate standard error. (c) gross appearance of hydrogel slabs after modified Lowry assay in situ showing characteristic blue color with starting peptide concentration (mmol/mL). The linear dependence on concentration was also valid in solid hydrogels (inset, bars indicate standard deviation). (d) The assay tracked CGRGDS retention over time within hydrogels. A large percent of RGDS was lost on the first day during hydrogel equilibrium swelling. The remaining peptide was stable for at least 2 more days in the gel (n=3 for all samples), with up to 75% retention. Bars indicate standard deviation. (e) HUVEC morphology on PEGDA hydrogels with 4.0 μmol/mL PEG-CGRGES (top) or PEG-CGRGDS (bottom) 24 h post-seeding. Scale bars=25 mm.

FIG. 5 presents (a) Representative images of chick aortic arch ring explants sprouting into hydrogels over time. In 8-wt % gels with 1.0 μmol/mL CGRGDS density, angiogenic sprouting varies with the MMP-susceptibility of the hydrogel backbone. No detectable sprouting occurred in negative control hydrogels containing RGES instead of RGDS peptide. Scale bar for all images=250 mm (b) Quantification of sprout area at Day 4, n=6 per condition. Mean with standard deviation, all comparisons are significant, p<0.003 by one-way ANOVA and Tukey's HSD post-hoc testing. (c) Fluorescent staining with lectin-rhodamine implicates endothelial cells as a principal component of the angiogenic sprouts in these hydrogels. Scale bar=100 mm (d) Composite image of selected frames during sprouting time-course by dark field imaging, false colored then overlaid here to aid in time visualization. Blue, yellow, orange, red=48, 62, 74, 86 h respectively. Scale bar=250

### DETAILED DESCRIPTION OF ILLUSTRATIVE EMBODIMENTS

The present invention may be understood more readily by reference to the following detailed description taken in connection with the accompanying Figures and Examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of any claimed invention. Similarly, any description as to a possible mechanism or mode of action or reason for improvement is meant to be illustrative only, and the invention herein is not to be constrained by the correctness or incorrectness of any such suggested mechanism or mode of action or reason for improvement. Throughout this text, it is recognized that the descriptions refer both to the method of preparing such devices and to the resulting, corresponding physical devices themselves, as well as the referenced and readily apparent applications for such devices.

In the present disclosure the singular forms “a,” “an,” and “the” include the plural reference, and reference to a particular numerical value includes at least that particular value, unless the context clearly indicates otherwise. Thus, for example, a reference to “a material” is a reference to at least one of such materials and equivalents thereof known to those skilled in the art, and so forth.

When values are expressed as approximations, by use of the antecedent “about,” it will be understood that the particular value forms another embodiment. In general, use of the term “about” indicates approximations that can vary depending on the desired properties sought to be obtained by the disclosed subject matter and is to be interpreted in the specific context in which it is used, based on its function, and the person skilled in the art will be able to interpret it as such. Where present, all ranges are inclusive and combinable.

It is to be appreciated that certain features of the invention which are, for clarity, described herein in the context of separate embodiments, may also be provided in combination in a single embodiment. Conversely, various features of the invention that are, for brevity, described in the context of a single embodiment, may also be provided separately or in any subcombination. Further, references to values stated in ranges include each and every value within that range.

Generally terms are to be given their plain and ordinary meaning such as understood by those skilled in the art, in the context in which they arise. To avoid any ambiguity, however, several terms are described herein.

The disclosures of each patent, patent application, and publication cited or described in this document are hereby incorporated herein by reference, in their entirety.

The present invention provides an inexpensive, flexible, and readily available route to bioactive PEG-based hydrogels, which can modulate ex vivo angiogenic sprouting through chemical control of MMP-susceptibility. In the first stage of synthesis, we used the step-growth polymerization of bis-cysteine MMP-sensitive peptides and bifunctional PEG compounds such as PEG-diacrylate (PEGDA) and PEG-diacrylamide (PEGDAAm) to make high molecular weight (MW) photoactive macromers. These macromers were then crosslinked into hydrogels during a second radical-mediated photopolymerization step. Under the conditions described, the synthetic scheme yields polydisperse materials with a majority of molecular species greater than 500 kDa. The presence of terminal acrylate or acrylamide groups permits photopolymerization via standard techniques, and the resultant hydrogels were highly susceptible to collagenase-mediated degradation. A peptide quantification assay was designed and employed to verify the amount of cell-adhesive peptide covalently incorporated into these hydrogels. These materials were then applied to examine, for the first time, 3D angiogenic sprouting from an ex vivo chick aortic arch assay into wholly synthetic materials. Angiogenic sprouts contained endothelial cells, and the sprouting response depended on both the MMP-susceptibility of the hydrogel backbone and the presence of adhesive peptide (CGRGDS compared to CGRGES). The control of angiogenic sprouting demonstrated here through modification of MMP-susceptibility alone highlights the general power of a synthetic approach to isolate a single parameter that in a natural scaffolding cannot be controlled independently from other properties. Specifically, this work may provide a new avenue to promote blood vessel growth in synthetic materials for tissue engineering and cell biology applications.

In some embodiments, this new technology produces macromers 500-3,000 kDa or more, which enables large mesh size for proper angiogenesis, but can be broken down into smaller starting material for proper clearance. Furthermore the manufacturing of this hydrogel is considerably cheaper and more reproducible than other methods, and the manufactured hydrogels are more stable than other options.

The hydrogels of the instant invention can be used in the design and production of tissue engineered scaffolds for a variety of therapeutic purposes including organ replacement, and in the musculoskeletal, cardiovascular, orthopedic, and neurological systems. Hydrogels can be made ex vivo and transplanted into the body, or injected as a liquid and polymerized into a solid hydrogel while inside the body. Hydrogels can also be commercialized for research purposes.

The MMP sensitive peptides are incorporated to allow for cells to naturally degrade and remodel the environment, which influences the cellular function in the scaffold. The high molecular weight of the macromers allows for the hydrogel to have large mesh size to enable proper angiongenesis and cellular migration and spreading. These macromers can then break down to be properly cleared by the liver and kidneys. The synthetic approach presented here highlights the potential utility of PEG-based hydrogels to support and control angiogenesis. Further, angiogenesis was found to be dependent on the MMP sensitivity of the hydrogel backbone, highlighting the ability to fully control vascularization by altering only one parameter in the synthesis of the scaffold.

Suitable polymer (P) that are useful in the instant invention include polyethylene glycol (PEG), alginate, polyurethane, and polyvinyl alcohol. In some embodiments, P refers to an oligomer or polymer with a molecular weight of 500-200,000 and in some embodiments has a molecular weight of 1,000 to 10,000.

As used herein, poly(ethylene glycol) refers to an oligomer or polymer of ethylene oxide of the formula HO—CH₂—(CH₂—O—CH₂)ₙ—CH₂—OH. In some embodiments, PEG has a molecular mass below 20,000 g/mol.

Alginate is an is an anionic polysaccharide which is commonly used as a hydrogel material. Cross-linking of alginate can be accomplished by means well known to those skilled in the art.

Polyurethane is any polymer consisting of a chain of organic units joined by urethane (carbamate) links. Typically polyurethanes are made from reacting diisocyanates (such as toluene diisocyanate (TDI) or diphenylmethane diisocyanate (MDI)) with a polyol (such as for example, polyether or polyester polyols having a molecular weight of 500 to 10,000).

Polyvinyl alcohol (PVA) is a polymer or oligomer of vinyl alcohol. In some embodiments, PVA has a molecular weight of 10,000 to 190,000.

As used herein, unless otherwise specified, molecular weight (MW) refers to weight average molecular weight. Molecular weight can be determined by methods well known to those skilled in the art.

Matrix metalloproteinase (MMP)-sensitive protein are defined as a protein or peptide sequence able to be cleaved at one or more sites by one or more members of the matrix metalloproteinase family such as, but not limited to, MMP-1. In some embodiments, the MMP is MMP-1, MMP-2, MMP-8, MMP-9, MMP-13, MMP-14, and MMP-18.

A wide variety of proteins, including enzymes and peptides may be used in constructing the macromers. Some preferred peptides include CGRGDS, CGRGES, CGPQGIAGQGCR, CGPQGPAGQGCR or CGPQGIWGQGCR. In some embodiments, the protein is a bis-cysteine matrix metalloproteinase (MMP)-sensitive peptide comprises at least one of CGPQGIAGQGCR, CGPQGPAGQGCR and CGPQGIWGQGCR. In certain embodiments, MMP-sensitive peptides can be used in combination with non-MMP sensitive peptides.

### Measurement of Mechanical Tractions Exerted by Cells in Three-Dimensional Matrices

An additional aspect of the invention concerns use of the hydrogels to form 3D matrices for cell growth. Cells are constantly probing, pushing and pulling on the surrounding extracellular matrix. These cell-generated forces drive cell migration and tissue morphogenesis, and maintain the intrinsic mechanical tone of tissues (Dembo, M. & Wang, Y. L. Biophys. J. 76, 2307-2316 (1999) and Keller, R., Davidson, L. A. & Shook, D. R. Differentiation 71, 171-205 (2003)). Such forces not only guide mechanical and structural events but also trigger signaling pathways that promote functions ranging from proliferation to stem-cell differentiation. Therefore, precise measurements of the spatial and temporal nature of these forces are essential to understanding when and where mechanical events come to play in both physiological and pathological settings.

Methods using planar elastic surfaces or arrays of flexible cantilevers have been used to map, with subcellular resolution, the forces that cells generate against their substrates (Dembo, M. & Wang, Y. L. Biophys. J. 76, 2307-2316 (1999); Balaban, N. Q. et al. Nat. Cell Biol. 3, 466-472 (2001); Butler, J. P., Tolic-Norrelykke, I. M., Fabry, B. & Fredberg, J. J. Am. J. Physiol. Cell Physiol. 282, C595-C605 (2002); and Tan, J. L. et al. Proc. Natl. Acad. Sci. USA 100, 1484-1489 (2003)). But many processes are altered when cells are removed from native three-dimensional (3D) environments and maintained on two-dimensional (2D) substrates. Cells encapsulated in a 3D matrix exhibit dramatically different morphology, cytoskeletal organization and focal adhesion structure from those on 2D substrates (Cukierman, E., Pankov, R., Stevens, D. R. & Yamada, K. M. Science 294, 1708-1712 (2001)). Even the initial means by which cells attach to and spread against a 2D substrate are quite different from the invasive process required for cells to extend inside a 3D matrix. These differences suggest that dimensionality alone may substantially impact how cellular forces are generated and transduced into biochemical or structural changes. Although the mechanical properties of 3D extracellular matrices and the cellular forces generated therein have been shown to regulate many cellular functions 9, to our knowledge, cellular forces in a 3D context have yet to be quantitatively measured.

Here we quantitatively measure the traction stresses (force per area), hereafter referred to as ‘tractions’, exerted by cells embedded in a hydrogel matrix. We encapsulated enhanced GFP (EGFP)-expressing fibroblasts in mechanically well-defined polyethylene glycol (PEG) hydrogels that incorporate proteolytically degradable domains in the polymer backbone and pendant adhesive ligands (Miller, J. S. et al. Biomaterials 31, 3736-3743 (2010)). Incorporation of adhesive and degradable domains permitted the cells to invade, spread and adopt physiologically relevant morphologies. The hydrogels used in this study had a Young's modulus of 600-1,000 Pa, a range similar to that of commonly used extracellular matrices such as reconstituted collagen or Matrigel and to in vivo tissues such as mammary and brain tissue (Paszek, M. J. et al. Cancer Cell 8, 241-254 (2005) and Discher, D. E., Janmey, P. & Wang, Y. L. Science 310, 1139-1143 (2005)). Cells in 3D PEG gels deformed the surrounding matrix, which we visualized by tracking the displacements of 60,000-80,000 fluorescent beads in the vicinity of each cell. We determined bead displacements relative to a reference stress-free state of the gel after lysing the cell with detergent. Typically we observed deformations of 20-30% peak principal strain in much of the hydrogel surrounding the cell. The largest strains, up to 50%, occurred in the vicinity of long, slender extensions, which is consistent with observations of strong forces exerted by these regions on 2D substrates (Chan, C. E. & Odde, D. J. Science 322, 1687-1691 (2008)). Because the mechanics of the PEG hydrogels showed no substantial dependence on strain or frequency, we used linear elasticity theory and the finite element method to determine the cellular tractions that would give rise to the measured bead displacements. Briefly, we generated a finite element mesh of the hydrogel surrounding the cell from confocal images. We constructed a discretized Green's function by applying unit tractions to each facet on the surface of the cell mesh and solving the finite element equations to calculate the induced bead displacements. Standard regularization methods for ill-posed, overdetermined linear systems of equations were then used to compute the tractions exerted by the cell. The time required to calculate a single dataset was ˜4.5 h using readily available computational equipment. However, we could reduce this dramatically by using a simplified finite element mesh of the cell and hydrogel. These lower-resolution datasets still captured the fundamental character of higher-resolution measurements.

To validate the approach and to characterize its spatial resolution, we used simulated traction fields. We measured experimental noise owing to bead displacements in cell-free regions of the hydrogel before and after detergent treatment, and measured surface discretization noise from multiple discretizations of the same cells. Then we superimposed these datasets onto the displacements generated by simulated loadings before traction reconstruction. In this setting, the percentage of traction recovered was proportional to the magnitude and characteristic length of the simulated loadings (defined as the average period of spatial oscillation). For all cases, the presence of noise reduced recovery accuracy by ˜20-30%. Despite these limitations, the recovered tractions still captured the essential periodic features of even the most spatially complex simulated loadings with characteristic lengths of spatial variation down to 10 μm.

We next calculated the tractions from live cells encapsulated in 3D hydrogels and found that cells exerted 100-5,000-Pa tractions, with strong forces located predominantly near the tips of long, slender extensions. For all measurements, forces were in static equilibrium with a typical error of ˜1-5% of the total force applied by the cell. Subsequent analysis revealed that these tractions were minimally impacted by possible variations in local hydrogel mechanics or by uncertainty in the measured bead displacements. Previous measurements of cellular forces on 2D surfaces have generally been limited to shear loadings, although recent studies have measured small forces exerted normal to the planar surface as well (Maskarinec, S. A., Franck, C., Tirrell, D. A. & Ravichandran, G. Proc. Natl. Acad. Sci. USA 106, 22108-22113 (2009) and Hur, S. S., Zhao, Y., Li, Y. S., Botvinick, E. & Chien, S. Cell. Mol. Bioeng. 2, 425-436 (2009)). It is unclear, however, whether these relationships might be altered for cells inside a 3D matrix. Here we found that cells encapsulated in a 3D matrix predominantly exerted shear tractions, although small normal tractions were also present near the cell body. To determine whether patterns of force might be associated with specific cell regions, we quantified the magnitude and angle of tractions with respect to the center of mass of the cell. Generally, tractions increased as a function of distance from the center of mass. Cells encapsulated in hydrogels with a Young's modulus of ˜1,000 Pa generated stronger tractions than those in ˜600-Pa hydrogels. The observed differences in tractions were not due to an overall increase in total cellular contractility, as measured by the net contractile moment but rather were most apparent in strong inward tractions near the tips of long, slender extensions. This reveals a local and nonlinear reinforcement of cellular contractility in response to substrate rigidity and suggests that such regions may be hubs for force-mediated mechanotransduction in 3D settings. The cell bodies showed no bias in traction angle, but strong tractions became progressively aligned back toward the center of mass in more well-spread regions of the cell (for example, near the tips of long, slender extensions). In general, these patterns of force were reflected in multiple cell types but could be altered by cell-cell proximity or maintenance as a multicellular aggregate. Neighboring NIH 3T3 cells preferentially extended away from each other, whereas proliferating multicellular tumor spheroids exerted outward normal tractions on the matrix.

Upon closer inspection we found a subset of extensions that displayed strong tractions several micrometers behind the leading tip, whereas the tractions at the tip itself were substantially lower. As such traction profiles are similar to those observed behind the leading edge of a lamellipodia for a migrating cell on a 2D substrate (Dembo, M. & Wang, Y. L. Biophys. J. 76, 2307-2316 (1999)), we hypothesized that such regions may represent invading or growing cellular extensions in three dimensions. To test this possibility, we measured the tractions from time-lapse images of cells as they invaded the surrounding hydrogel. Indeed, tractions at the tips of growing extensions were notably lower than the strong tractions exerted by proximal regions of the same extension. However, we did not observe normal forces pushing into the extracellular matrix in these extensions, which suggests that a local inhibition of myosin-generated contractility allows tip advancement. Moreover, we also detected strong tractions from small extensions on the cell face opposite the invading extensions. Such stable extensions exhibited very different force distributions than the growing extensions, often lacking the characteristic drop in force near the leading edge, and may correspond to an anterior-posterior polarity axis formed in the cell.

These data suggest that cells in 3D matrices probe the surrounding extracellular matrix primarily through strong inward tractions near the tips of long, slender extensions. This technique was generalizable to different cell types, cell-cell interactions and even to multicellular tumor structures in which both tumor growth and invasion have been previously shown to be mechanoresponsive (Paszek, M. J. et al. Cancer Cell 8, 241-254 (2005)). Because the synthetic hydrogels used in this study had similar elastic moduli to in vivo tissues (Paszek, M. J. et al. Cancer Cell 8, 241-254 (2005) and Discher, D. E., Janmey, P. & Wang, Y. L. Science 310, 1139-1143 (2005)) and can support many cellular functions (Lutolf, M. P. & Hubbell, J. A. Nat. Biotechnol. 23, 47-55 (2005)), this approach should enable investigations into the role of cellular forces in various biological settings.

The invention is illustrated by the following examples which are intended to be illustrative and not limiting.

### Materials and Methods

### Reagents and Cell Maintenance

All reagents were from Sigma-Aldrich (St. Louis, Mo.) and were used as received unless otherwise described. Acryloyl chloride was from Alfa Aesar (Ward Hill, Mass.). Culture media and human umbilical vein endothelial cells (HUVECs) were from Lonza (Basel, Switzerland), and were maintained in complete Endothelial Growth Medium-2 (EGM-2, Lonza).

Synthesis and Characterization of Poly(ethylene glycol) Diacrylate (PEGDA)

Dry poly(ethylene glycol) (PEG; MW 3400 or 6000) was acrylated by reaction with triethylamine (TEA; clear, colorless, 2 molar excess to PEG) and acryloyl chloride (clear, colorless, 4 molar excess to PEG) in anhydrous dichloromethane under argon as described previously 1 Mann, et al., Biomaterials. 2001; 22:3045-511. Yields were typically in the range 80-90% (˜120 g), and percent acrylation was 99% as verified by ¹H NMR for the characteristic peak (4.32 ppm) of the PEG methylene protons adjacent to the acrylate 1 Mann, et al., Biomaterials. 2001; 22:3045-511.

### Synthesis and Characterization of Poly(Ethylene Glycol) Diacrylamide (PEGDAAm)

Polyethylene glycol diacrylamide (PEGDAAm; MW, 3400) was synthesized from PEG by forming the dimesylate, then the diamine and finally the diacrylamide as described previously (Elbert, D. L. & Hubbell, J. A. Biomacromolecules 2, 430-441 (2001)).

### Synthesis of MMP-Sensitive Acrylate-PEG-(Peptide-PEG)ₘ-Acrylate Conjugates

The bis-cysteine peptide sequences CGPQGIWGQGCR (highly degradable, HD, 1261.42 g/mol), CGPQGIAGQGCR (native collagen, NC, 1146.28 g/mol), and CGPQGPAGQGCR (least degradable, LD, 1130.23 g/mol) were custom synthesized by Aapptec (Louisville, Ky.). Each peptide was supplied as a trifluoroacetate salt at >95% purity. Peptides were evacuated of air and stored under argon (to minimize disulfide formation) at −80° C. until needed. In a typical reaction, 183.8 μmol bis-cysteine peptide (HD, 231.6 mg) was reacted with a 1.6 molar excess of PEGDA (3400 Da, 1 g, 294.1 μmol) by dissolution in 10 mL 100 mM sodium phosphate, pH 8.0 (94.7 mM Na₂HPO₄, 5.3 mM NaH₂PO₄). The reaction was sterile filtered through a 0.22 μm PVDF membrane (Millipore, Billerica, Mass.), protected from light and proceeded on a circular shaker for 85 hr at room temperature to yield acrylate-PEG-(peptide-PEG)ₘ-acrylate conjugates. The reaction mixture was dialyzed against 4 L 18 MΩ water (Millipore) with pre-swollen regenerated cellulose dialysis tubing (MWCO 3500, “snake-skin”, Pierce, Rockford, Ill.) for 24 hr (4 water changes). The dialyzed PEG-peptide conjugates were frozen overnight (−20° C.), lyophilized, and stored at −80° C. until use.

### Characterization of PEG-Peptide Macromers by GPC

PEG-peptide conjugates were analyzed by GPC with a refractive index detector and DMF solvent using three tandem styrene-divinylbenzene (SDVB) columns spanning a linear MW range from 1 kDa to 500 kDa for polystyrene. PEG MW standards from 628 Da to 478 kDa (Sigma) were used for assessment of the molecular weight of the PEG-peptide conjugates. Columns spanning a larger MW range, into the tens of MDa range, may enable more complete characterization of larger MW macromers synthesized.

### Synthesis of MMP-Sensitive Vinylsulfone-PEG-(Peptide-PEG)ₘ-Vinylsulfone and Acrylamide-PEG-(Peptide-PEG)ₘ-Acrylamide Conjugates

Vinylsulfone-PEG-(peptide-PEG)ₘ-vinylsulfone macromers or acrylamide-PEG-(peptide-PEG)ₘ-acrylamide macromers are synthesized as described above for the synthesis of acrylate-containing macromers but can safely utilize stronger aqueous base solutions such as 0.1 N NaOH, 100 mM sodium borate pH 9.0, or similar solutions known to those skilled in the art during macromer synthesis. The reaction may be carried out under these conditions for 1-60 days depending on the reaction efficiency and target molecular weight and polydispersity desired. Products are monitored during synthesis and characterized following synthesis by GPC.

### Synthesis of Macromers Using Organic Solvent

1.6 molar equivalents of PEGDA or PEGDAAm or PEG-divinylsulfone per mole of the bis-cysteine peptide CGPQGIWGQGCR were dissolved in toluene and evaporated to a thick oil. The evaporated oil was dissolved in dimethylformamide to bring the concentration of PEG polymer to 50 mg/mL. In an alternate synthesis, 1.6 molar equivalents of PEG-di-N-hydroxysuccinimide (PEG-di-NHS) per mole of the diamine peptide GPQGIWGQK were dissolved in toluene and evaporated to a thick oil. The evaporated oil was dissolved in dimethylformamide to bring the concentration of PEG-di-NHS to 50 mg/mL. The peptides to be PEGylated were added along with 1M equivalent of triethanolamine per mole of matched polymer species as described above and reacted between 4 hrs-60 days specific for the individual reaction scheme. Bis-cysteine peptides are reacted with one or more of PEGDA, PEGDAAm, PEG-divinylsulfone to yield acrylate-PEG-(peptide-PEG)ₘ-acrylate, acrylamide-PEG-(peptide-PEG)ₘ-acrylamide, or vinylsulfone-PEG-(peptide-PEG)ₘ-vinylsulfone respectively. In the alternate strategy, diamine peptides are reacted with PEG-di-NHS to yield NHS-PEG-(peptide-PEG)ₘ-NHS. The product was precipitated in ether and dried, then dissolved in diH₂O, sterile filtered, and purified by dialysis. Products were lyophilized and stored at −20° C.

PEG-Peptide Macromer Photopolymerization to form Hydrogels

PEGDA or PEG-peptide macromers were individually dissolved at 8-20% w/w concentration in PBS to make stock prepolymer solutions at the beginning of each experiment. The desired amounts of cell-adhesive and MMP-sensitive macromers were then mixed and diluted to the proper experimental concentration with PBS. To maintain concentration accuracy during dissolution, it was noted that PBS volume increased upon addition of PEG-peptide conjugates by approximately 0.9 μL/mg added. All macromers are reported as their initial concentration during hydrogel polymerization. A solution (100 mg/mL in 100% ethanol) of the photoinitiator Irgacure 2959 (I2959, Ciba, Tarrytown, N.Y.), was added to a final working concentration of 0.05% w/v (by using 5 μL of the initiator solution per 1 mL hydrogel prepolymer solution). Solutions were thoroughly mixed and sonicated before polymerization. The prepolymer solution was transferred into plastic molds (96-well plate) for degradation assays, between glass plates for the modified Lowry assay, or dispensed onto a sterile slab of poly(dimethyl siloxane) (PDMS; Dow Corning) for explant encapsulation as described below. Photopolymerization was conducted with an Omnicure S2000 (320-500 nm, EXFO, Ontario, Canada) lamp at 100 mW/cm² (measured for 365 nm) to yield solid hydrogels (exposure times reported in relevant sections below). Hydrogels containing explants were easily transferred into culture media with flat, round tip tweezers (EMS, Switzerland).

### Characterization of MMP-Sensitive PEG-Peptide Hydrogels by Collagenase Degradation

A collagenase degradation assay was employed to check the MMP-sensitivity of these hydrogels and their relative degradation behavior, in a similar fashion as described previously Mann, et al., Biomaterials. 2001; 22:3045-511. Briefly, hydrogel prepolymer solutions were made in HEPES-buffered saline (HBS; 10 mM, pH 7.4) containing 0.2 mg/mL sodium azide (to inhibit microbial growth), mixed with initiator, and polymerized for 60 sec as described above. Hydrogels (150 μL starting volume per gel) were swollen for 36 hr at 37° C. and weighed to assess equilibrium swollen weight. These swollen hydrogels were then transferred to a 0.2 mg/mL collagenase solution (made with the same buffer) and their wet weight was monitored over time (3 gels per condition). Control hydrogels were incubated in buffer without enzyme.

### Synthesis and Characterization of Cell-Adhesive Acrylate-PEG-Peptide Conjugates

Cell adhesive or non-adhesive acrylate-PEG-peptide conjugates were prepared in a similar manner to the MMP-sensitive conjugates by using a 1.0 molar equivalent of PEGDA 3400 for the monocysteine peptides CGRGDS (adhesive, 593.59 g/mol) and CGRGES (non-adhesive, 607.62 g/mol). These conjugates were characterized by GPC as described above.

### Characterization of the Immobilization Stability of Cell-Adhesive Acrylate-PEG-Peptide Conjugates

To verify the immobilization stability of acrylate-PEG-RGDS in PEG gels we developed a modified Lowry Assay (Sigma) in prepolymer solutions or in solid hydrogels to quantify peptide concentration in situ. For solutions, acrylate-PEG-CGRGDS solutions were made in sterile water (the Lowry assay is not reliable in PBS) and assessed as described below with the free peptide CGREDV used as a standard. For solid hydrogels, 10% w/w PEGDA 6000 hydrogel prepolymer solutions were made containing 0, 0.25, 2, or 4 μmol/mL acrylate-PEG-CGRGDS. Initiator was added as described above, then each solution was transferred to a glass chamber composed of thin rubber spacers sandwiched between two glass slides (chamber dimensions: 30 mm×40 mm×0.48 mm thick). Hydrogels were polymerized for 120 seconds (25 mW/cm²) and then sliced into 3 sections to yield hydrogels approximately 7 mm×15 mm×0.48 mm Gels were subjected to a modified Lowry assay immediately after polymerization, or after a 24 hr or 72 hr incubation at 37° C. in sterile water (changed daily). At these specified times, hydrogels were blotted dry with laboratory wipes, then placed in a test tube with 1 mL deionized water. While vigorously mixing, 1 mL Lowry reagent was added according to the vendor's recommendations. Mixing continued for 40 sec and hydrogels were left at room temperature for 20 min. While vigorously mixing, 0.5 mL Folin-Ciocalteu's phenol reagent was added. Mixing continued for 40 sec and hydrogels were left at room temperature for 30 min Hydrogels were blotted dry, transferred to plastic cuvettes and assessed with a UV/Vis spectrophotometer (750 nm) transverse to the wide hydrogel face. Absorbance values were normalized to PEGDA gels without peptide.

### Characterization of Cell Attachment to Adhesive Acrylate-PEG-Peptide Conjugates

Cell-adhesive 20% w/w PEGDA 3400 hydrogels were formed containing 4 μmol/mL acrylate-PEG-CGRGDS or acrylate-PEG-CGRGES in PBS and swollen for 24 hr at 37° C. Hydrogels were briefly rinsed with media, then seeded with HUVECs (15,000 cells/cm²). Hydrogels were rinsed with PBS after 24 hr and photographed to check cellular attachment.

### Chick Aortic Arch Explant Angiogenesis Assay

Chick aortas were isolated from 12-day-old chick embryos (Charles River Labs, Preston, Conn.). Aortic arches were cleaned of excess fibroadipose tissue, sectioned into ˜0.5 mm sized rings, and submerged inside a 30 μL droplet of hydrogel prepolymer solution (final concentrations of 8% w/w MMP-sensitive and 1.0 μmol/mL adhesive components). Polymerization was performed for 30 sec as described above, and culture media (EGM-2; 0.75 mL per hydrogel) was changed on day 1 and every 3 days thereafter. Hydrogels were photographed daily with oblique lighting phase contrast microscopy to optically exclude 2D cell migration on the surface of hydrogels and instead visualize only those cells which migrated in 3D within the hydrogels. Sprout area was assessed by image thresholding and edge-finding filters (Adobe Photoshop, NIH ImageJ), 2 sides per arch ring, 6 arch rings per experimental group. Statistics were assessed by one-way ANOVA with Tukey's HSD post-hoc testing, and p-values less than 0.05 were considered significant. For time-lapse microscopy, hydrogels containing arch pieces were polymerized on round coverslips (22 mm) that were functionalized with 3-(trimethoxysilyl)propyl methacrylate according to the manufacturer's instructions (Sigma) to covalently link the hydrogel to the glass coverslip. Briefly, coverslips were sonicated in Alconox detergent, rinsed with 18 M52 water, blown dry with nitrogen, and baked at 110° C. for 30 min. Cleaned and dried coverslips were then placed in a 2% v/v solution of the silane in EtOH (200 mL) with dilute acetic acid (6 mL, 1:10 glacial acetic acid:water) at room temperature for 1 hr, blown dry with nitrogen, then baked at 60° C. for 1 hr. Hydrogel prepolymer solutions (20 μL) were placed into PDMS wells on these coverslips and photopolymerized as described above. After 2 days in culture, these gels were mounted on an environmentally controlled microscope (5% CO₂, 37° C.; Zeiss Axiovert 200M, Carl Zeiss, Germany) and imaged by oblique lighting phase contrast every hour. For endothelial cell labeling experiments, aortic arches explants were incubated with rhodamine-lectin (Lens culinaris agglutinin, 20 μg/ml, Vector Laboratories) for 1.5 hr before encapsulation in hydrogels.

### Results and Discussion

### Macromer Design and Analysis

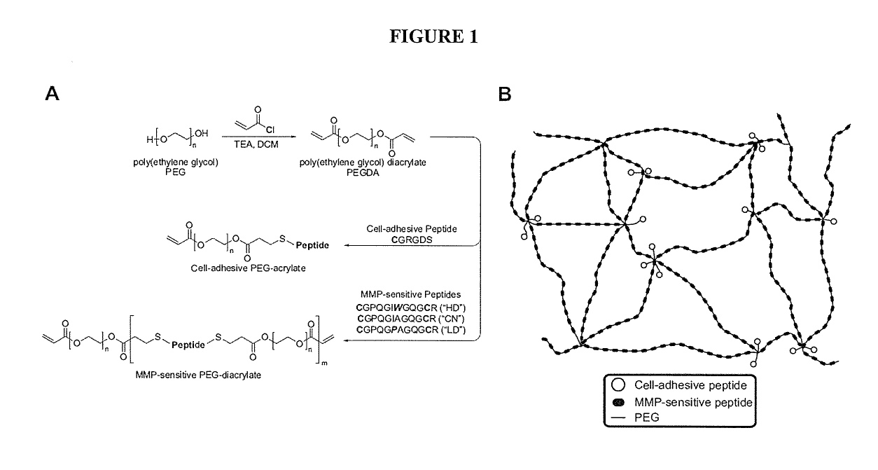

This work examines the step-growth polymerization of PEGDA with MMP-sensitive peptides for tissue engineering and cell biology applications. We started with synthesis of PEGDA from PEG, as previously described (FIG. 1a, Mann, et al., Biomaterials. 2001; 22:3045-51) Importantly, ensuring the clear and colorless properties of the starting reagents TEA and acryloyl chloride are critical to achieving a high percentage of acrylation. With pure reagents, this synthesis lends itself well to scale-up in the laboratory, with PEGDA batch yields routinely 120 g or greater (80-90% yield) and percent acrylation greater than 99%. Compared to other routes to bioactive PEG-based hydrogels, which employ acrylate-PEG-NHS Gobin, FASEB J. 2002; 16:751-3] or multi-arm PEGs Raeber, et al., Acta Biomater. 2007; 3:615-29 and Aimetti, et al., Biomaterials. 2009; 30:6048-54], our approach here is much less subject to proprietary restrictions, vendor sourcing or availability issues, or synthetic difficulties. Material cost for the current approach is also dramatically reduced for these simple PEGs (up to 100× based on current market rates). Indeed the entire range of readily available PEG molecular weights, from oligoethylene glycols to 100 kDa poly(ethylene oxide) should be amenable to this synthetic scheme. Keeping future in vivo targets in mind, PEG 3400 was chosen as the base structural unit for these hydrogels due to its well-known ability to be cleared in vivo. As with other synthetic approaches, we believe the current approach to be extremely flexible for examining a wide variety of matrix properties. In this work we examined the effects of hydrogel degradation rate on 3D angiogenic sprouting.

Our strategy employed an initial step-growth polymerization between PEG and peptides to yield soluble, high MW photoactive precursors. MMP-sensitive peptide sequences were selected based on a range of known degradabilities [Imper V, Van Wart HE. Substrate Specificity and Mechanisms of Substrate Recognition of the Matrix Metalloproteinases. A chapter in Matrix Metalloproteinases, edited by W C Parks and R P Mecham. 1998; Academic Press: 219-42], and previous work with this family of sequences in degradable hydrogels [Lutolf, et al., Nat. Biotechnol. 2003; 21:513-8 and Lee S-H, Miller J S, Moon J J, West J L. Proteolytically degradable hydrogels with a fluorogenic substrate for studies of cellular proteolytic activity and migration. Biotechnol Prog. 2005; 21:1736-41]. These base sequences were flanked with leading and lagging cysteine residues (FIG. 1a; HD=highly degradable, CN=collagen native, LD=least degradable) to allow for reaction with the terminal acrylates on PEGDA. Our use of PEGDA rather than multi-arm PEGs means that step-growth polymerization does not result in hydrogel formation directly, but rather leads to macromer chain extension such that multiple MMP-sensitive peptides are incorporated into each polymer chain (FIG. 1b). Sodium phosphate buffer pH 8.0 proved an effective buffer for macromer coupling because it is sufficiently basic to allow for Michael-type addition while still mild enough to leave the terminal ester bonds of PEGDA intact. Furthermore, disulfide bonding is not favored under these conditions [Lutolf and Hubbell, Biomacromolecules. 2003; 4:713-22]. The resulting high MW macromers could then be purified and reconstituted in phosphate buffered saline (PBS), and crosslinked in the presence of living cells to form bioactive hydrogels in a second rapid photopolymerization step (FIG. 1b). We found the main characteristics of this unique system to be increased hydrogel swelling and collagenase sensitivity, and dramatically decreased material cost, compared to other synthetic strategies for PEG-based gels.

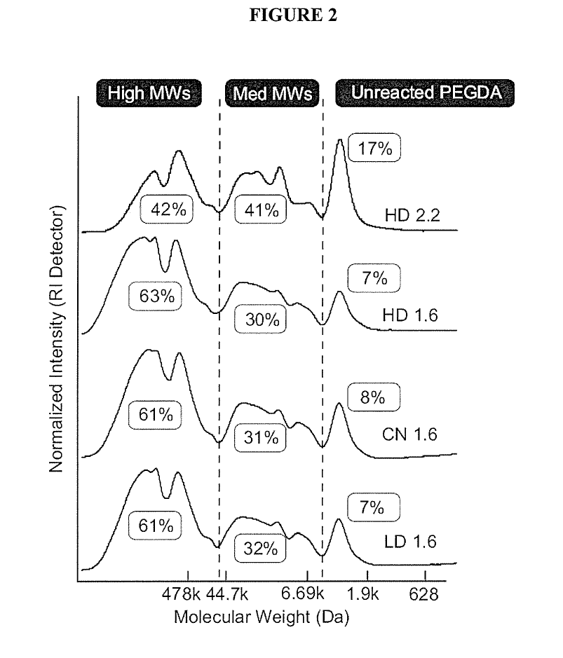

Step-growth polymerization is strongly controlled by the stoichiometric ratio of the reactants, and we found large differences in resultant polydispersity based on the starting ratio of PEGDA:peptide used for each reaction (FIG. 2). In order to ensure that acrylates remained at the terminal ends of MMP-sensitive macromers (to enable later photopolymerization), an excess of PEGDA compared to peptide was used. With a PEGDA:peptide molar ratio of 2.2, more than 80% of the PEGDA reacted with peptide (sum of “high” and “medium” MWs in FIG. 2) indicating successful Michael-type addition. Surprisingly, approximately 40% of the resultant molecular species were greater than 500 kDa. Unreacted MMP-sensitive peptide was not observed by GPC, either due to the completeness of the reaction or from being washed away during dialysis.

To achieve higher coupling efficiency, a PEGDA:peptide ratio of 1.6 was used (FIG. 2). In this case, more than 90% of the PEGDA reacted with peptide and approximately 60% of the molecular species were greater than 500 kDa Importantly, all three MMP-sensitive peptides showed nearly identical polydispersity, indicating that Michael-type addition proceeded similarly for each peptide sequence. Reacted species are of the form acrylate-PEG-(peptide-PEG)ₘ-acrylate, and for a macromer MW of 500 kDa, the m-value is approximately 100.

To make pendant cell-adhesive RGDS peptide, we reacted CGRGDS peptide with PEGDA 3400 under similar conditions but with a PEG:peptide ratio of 1.0. GPC analysis showed that 87% of the PEGDA reacted with peptide. The lack of a second cysteine residue on this peptide prevents step-growth polymerization and thus the possibility of high MW macromers. However, double conjugation in the form peptide-PEG-peptide is possible in this reaction. Of the peptide-conjugated PEGDA, 63% was in the preferred acrylate-PEG-CGRGDS form. These data suggested sufficient coupling of peptide for their covalent incorporation into hydrogels as cell-adhesive pendant chains.

### Hydrogel Degradation in Collagenase

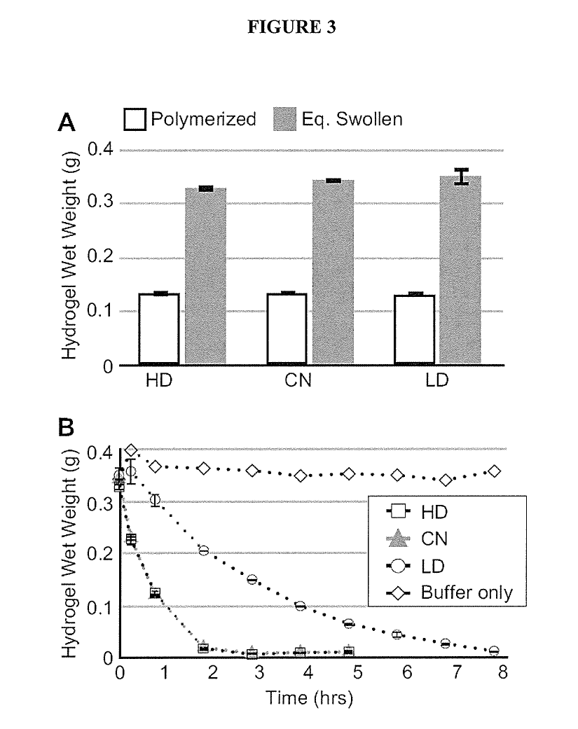

Step-growth derived macromers were photopolymerized into hydrogels, which were allowed to reach equilibrium swelling in aqueous buffer and then degraded in 0.2 mg/mL collagenase while their wet-weight was monitored. Buffer without collagenase served as negative control. Hydrogels absorbed a large amount of buffer solution during equilibrium swelling, with 10 wt % gels gaining a factor of 2.5× of their as—polymerized weight (FIG. 3a). This compares with a factor of 1.2-1.4× equilibrium swelling weight gain as reported for similar hydrogels [Mann, et al., Biomaterials. 2001; 22:3045-51 and Lee, et al., Biotechnol Prog. 2005; 21:1736-41]. The dramatic equilibrium swelling of these hydrogels is due to the high MW of the macromers in the hydrogel pre-polymer solution. The highly swollen nature of these gels, and the presence of multiple degradable peptides within each macromer chain were likely the principal contributors to their rapid degradation, with all gels fully degrading within 8 hr (FIG. 3b). These observed hydrogel degradation profiles differ substantially from the degradabilities reported for these sequences when in soluble form. Relative to the native collagen sequence (CN), reported degradabilities for HD and LD peptides in solution are 800% and 0.5%, respectively [Imper and Van Wart, HE. Substrate Specificity and Mechanisms of Substrate Recognition of the Matrix Metalloproteinases. A chapter in Matrix Metalloproteinases, edited by W C Parks and R P Mecham. 1998; Academic Press: 219-42]. In contrast, the degradation curves for hydrogels containing HD and CN peptides overlapped nearly identically. This overlap is likely a result of the concentration of collagenase used (0.2 mg/mL) which was selected to be consistent with the literature for these assays. Indeed, angiogenic sprouting assays (described below) indicate a significant difference between the degradable behaviors of these materials. Additionally, LD hydrogels required nearly twice the amount of time to fully degrade in collagenase compared to HD and CN gels. The difference in the reported degradabilities for soluble peptides compared to our observed degradation profiles for solid hydrogels may be attributed to the many repeating degradable peptides in the hydrogel backbone of the form acrylate-PEG-(peptide-PEG)ₘ-acrylate. Indeed, these hydrogels degrade extremely rapidly in collagenase compared to other MMP-sensitive hydrogels [Mann, et al., Biomaterials. 2001; 22:3045-51].

### Quantification of Acrylate-PEG-CGRGDS Immobilization and Assessment of Bioactive Potency

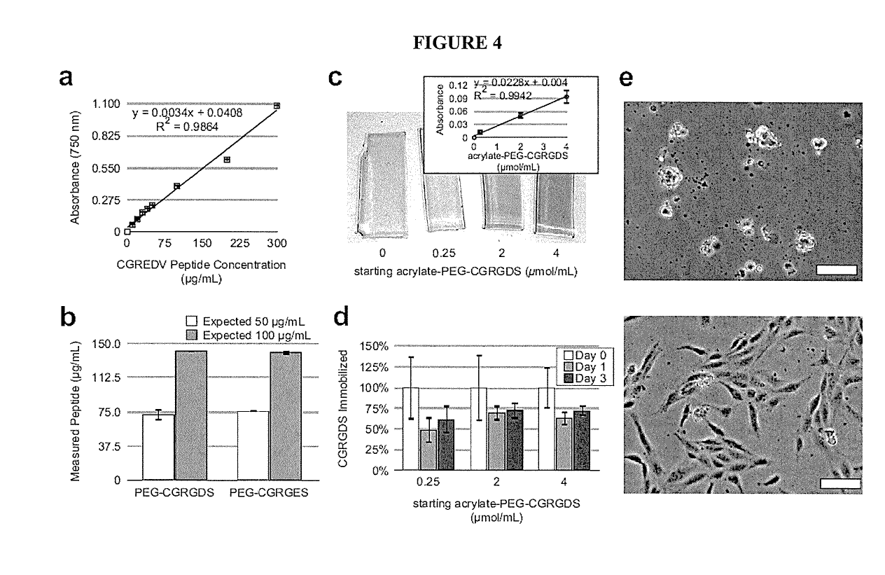

To enable cell-substrate adhesion in these MMP-sensitive hydrogels we employed the well-known RGDS peptide using a similar synthetic approach as that for step-growth polymerization (FIG. 1a). To quantify the amount of RGDS entrapped or immobilized in the hydrogel during polymerization and its subsequent stability in the hydrogel over time, we developed a modified Lowry Assay for in situ quantification [Lowry O H, Rosebrough N J, Farr A L, Randall R J. Protein measurement with the Folin phenol reagent. J Biol. Chem. 1951; 193:265-75]. Using this new modification, we were able to quantify the concentration and stability of immobilized adhesive peptide in hydrogels over time (FIG. 4a-d). The Lowry assay provides a colorimetric measurement of the total amount of peptide bonds present and is typically quantified relative to a bovine serum albumin (BSA) control. Because BSA was not a suitable standard for the short peptides employed here, which have comparatively fewer peptide bonds per mg of material, we first established the use of the short peptide CGREDV as a standard, which has the same number of peptide bonds as the peptides used in these experiments. Known amounts of CGREDV peptide were diluted in solution and quantified by Lowry assay. The resulting linear standard curve verified that the Lowry assay, typically used only for large proteins, could be used to quantify the concentration of short peptides (FIG. 4a). When this standard curve was applied to our acrylate-PEG-CGRGDS materials diluted in solution, we found an equivalence of peptide measured as expected for starting dry weight of the PEG-peptide conjugate with a deviation from expected of 1.4-1.5× (FIG. 4b). We then applied this assay to characterize the immobilization of CGRGDS into solid hydrogels. To remove the potentially confounding influence of degradable peptide immobilized in the gels, this assay was applied to non-degradable PEGDA hydrogels with or without adhesive ligand peptide. In solid hydrogel slabs, we again found a linear relationship between absorbance and peptide amount used (FIG. 4c), which validated this modified Lowry assay for solid hydrogels. To estimate the amount of peptide immobilized to the hydrogel, we followed relative peptide retention in hydrogels over time (FIG. 4d). In the first day of equilibrium swelling, hydrogels lost between 30-50% of the PEGDA-peptide conjugate. The remaining immobilized peptide was stable in the gel thereafter (FIG. 3b). These results are consistent with GPC analysis of the CGRGDS-conjugate, which indicated 63% in the preferred mono-conjugated acrylate-PEG-CGRGDS form. That is, double-conjugated peptide-PEG-peptide would initially be physically entrapped in the gel but would diffuse away during equilibrium swelling within the first day. These data therefore suggest that the preferred acrylate-PEG-CGRGDS species are largely covalently incorporated into the hydrogel. As stated in Materials and Methods, all adhesive macromers are reported as their initial concentration during hydrogel polymerization to aid in reproducing the results obtained here and to remain consistent with the existing literature. Moreover, this new modification of the Lowry assay may find uses in other hydrogel systems for verifying peptide immobilization and stability in situ.

We next confirmed the bioactive potency of our cell-adhesive conjugate using surface adhesion of human umbilical endothelial cells (HUVECs) to PEGDA hydrogels containing the cell-adhesive CGRGDS or non-adhesive CGRGES peptide (FIG. 4e). While negative control CGRGES peptide was unable to support HUVEC adhesion, CGRGDS peptide supported robust HUVEC adhesion and cell spreading. This assay provided an initial check of the bioactivity of our cell-adhesive conjugates, and confirms that sufficient adhesive PEG-peptide is immobilized in the hydrogels to support cell adhesion. Because HUVEC adhesion to PEG-based hydrogels containing RGDS peptide has been studied in detail elsewhere [Leslie-Barbick, et al., J Biomater Sci Polym Ed. 2009; 20:1763-79 and Moon J J, Hahn M S, Kim I, Nsiah B A, West J L. Micropatterning of poly(ethylene glycol) diacrylate hydrogels with biomolecules to regulate and guide endothelial morphogenesis. Tissue Eng Part A. 2009; 15:579-85], we instead focused on applying these conjugates to support three-dimensional studies of angiogenic sprouting.

### Aortic Arch Explant Assay

The possibility of using these materials to observe and control three-dimensional cell migration was examined with the chick aortic arch assay, in which angiogenic sprouting from embryonic chick explants (typically done in fibrin or collagen gels) is a reliable predictor of factors that stimulate angiogenesis in vivo [Auerbach R, Lewis R, Shinners B, Kubai L, Akhtar N. Angiogenesis assays: a critical overview. Clin Chem. 2003; 49:32-40 and Aplin A C, Fogel E, Zorzi P, Nicosia R F. The aortic ring model of angiogenesis. Meth Enzymol. 2008; 443:119-36]. While endothelial cells are activated into an angiogenic phenotype by numerous factors such as vascular endothelial growth factor (VEGF) [Adams R H, Alitalo K. Molecular regulation of angiogenesis and lymphangiogenesis. Nat Rev Mol Cell Biol. 2007; 8:464-78], their ability to form new vessels is likely also physically constrained and regulated by the interplay of cell-secreted MMPs with the extracellular matrix [Chun T-H, Sabeh F, Ota I, Murphy H, McDonagh K T, Holmbeck K, et al. MT1-MMP-dependent neovessel formation within the confines of the three-dimensional extracellular matrix. J. Cell Biol. 2004; 167:757-67 and Ghajar C M, George S C, Putnam A J. Matrix metalloproteinase control of capillary morphogenesis. Crit. Rev Eukaryot Gene Expr. 2008; 18:251-78]. To test this possibility, we used each of the three MMP-degradable sequences in our hydrogels to vary only MMP-susceptibility, while holding polymer weight percent and adhesive peptide concentration constant. Dark field imaging through oblique lighting phase contrast microscopy illuminated only cells within 3D angiogenic sprouts, allowing direct imaging and quantitation specifically of 3D sprouting.

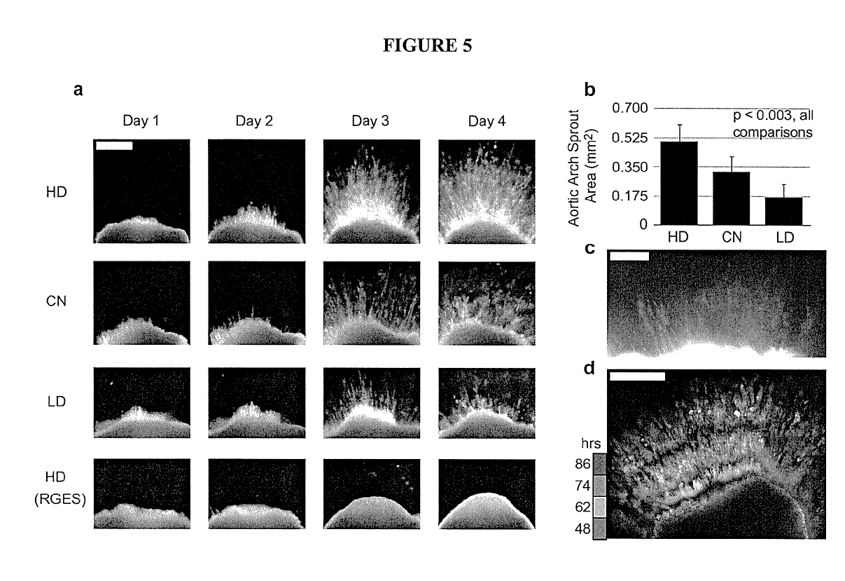

Significantly more 3D angiogenic sprouting was observed in the hydrogels containing the most degradable peptide sequences (FIG. 5a,b). Representative images demonstrate the character and time course of sprouting into these hydrogels. Quantification of area of sprouting from each explant demonstrates statistical significance between the three different experimental groups (p<0.003 for all comparisons by one-way ANOVA and post-hoc testing). Moreover, angiogenic sprouting was completely suppressed to undetectable levels by substitution of CGRGDS with the non-adhesive CGRGES peptide, confirming that the hydrogels support angiogenic invasion only in the presence of an adhesive peptide. To verify that the observed explant sprouts were of endothelial origin, we incubated the chick arches with rhodamine-conjugated Lens culinaris agglutinin lectin, which specifically labels endothelial cells [Mani S M, Murphy T J, Thai S N M, Eichmann A, Alva J A, Iruela-Arispe M L. Selective binding of lectins to embryonic chicken vasculature. J Histochem Cytochem. 2003; 51:597-604]. Indeed, endothelial cells were a principal component of the newly formed sprouts (FIG. 5c). Observation demonstrates a dark field time-course of angiogenic sprouting in these MMP-sensitive hydrogels. To visualize an angiogenic sprouting time-course in a single image we selected sequential movie frames 12-14 hours apart, false-colored them with time, and then overlaid them with no lateral translation (FIG. 5d).

### Hydrogel Synthesis and Cell Encapsulation.

PEGDAAm was then reacted with the collagenase-sensitive peptide in sodium borate (100 mM, pH 9.0) until the product polydispersity matched that for PEGDA-peptide precursors. For encapsulation, NIH 3T3 cells were resuspended to a final concentration of 60,000 cells ml-1 in a 10 or 11% (w/v) solution of degradable PEGDA-peptide macromer in PBS (pH 7.4) containing 1 μmol ml-1 acrylate-PEG-CGRGDS, 0.5 mg ml⁻¹ Irgacure 2959 (Ciba) and two types of fluorescent beads (0.2 μm diameter, nonfunctionalized, yellow-green dyed (Polysciences) and 0.2 μm diameter, nonfunctionalized, suncoast yellow dyed (Bangs Labs)) at ˜3.75×1010 beads ml⁻¹ each. Note that the pore size of the PEG gels was an order of magnitude smaller than the diameter of the beads used in this study18. Therefore, the beads were physically encapsulated in the hydrogel and did not diffuse. Bovine pulmonary artery smooth muscle cells, human mesenchymal stem cells and Lewis lung carcinoma cells were encapsulated in a 7% (w/w) solution of PEGDAAm-peptide macromer in PBS containing 5 μmol ml⁻¹ acrylate-PEG-CGRGDS, 5 μmol ml⁻¹ acrylate-PEG-CGRGES, 0.5 mg ml-1 Irgacure 2959 and two types of fluorescent beads. Next, 20 μl of cell-laden prepolymer solution was pipetted onto coverslips (0 thickness; Fisher Scientific) that were functionalized with 3-(trimethoxysilyl)propyl methacrylate (Sigma) per the manufacturer's instructions. The solution was contained in annular molds made from poly(dimethyl siloxane) (PDMS; Dow Corning) and exposed to 200 mW cm⁻² (measured at 365 nm) UV light from an Omnicure 52000 (320-500 nm; EXFO) for a total of 3,000 mJ. After removing the PDMS mold, polymerized hydrogels, which now formed a cylindrical disc that was ˜4 mm in diameter and 500 μm tall and were covalently linked to the coverslip along the bottom surface, were immersed in cell culture medium and incubated under standard growth conditions (37° C., 5% CO₂) for 72 h.

### Microscopy, Image Segmentation, Finite Element Mesh Generation and Computational Resources.

Encapsulated cells were imaged with a 40×, 1.1 numerical aperture (NA), water-immersion objective (LD C-Apochromat; Carl Zeiss) attached to an Olympus IX71 inverted microscope equipped with a CSU10 spinning disc confocal scan head (Yokogawa Electric Corporation), live-cell incubator (Pathology Devices) and an ImagEM 16-bit electron-multiplying charge-coupled device (EMCCD) camera (Hamamatsu Photonics). A 147×147×200 μm volume was imaged around each cell, which corresponded to voxel dimensions of 0.2841×0.2841×0.8 μm in both horizontal planes and the axial plane, respectively. After the stressed image was acquired, the cells were treated with 0.5% SDS (JT Baker), re-equilibrated for 45 min and then reimaged to acquire a reference image of the nonstressed hydrogel. This detergent was chosen so as to completely denature all cellular proteins, although in practice, more mild detergents or specific inhibitors of cytoskeletal contractility could be used as well. Time-lapse datasets were acquired at 30-min intervals and 1-μm spacing in the axial plane. This temporal and spatial resolution was chosen so as to increase the image acquisition speed (˜3 min of exposure per volume per cell) and to reduce phototoxicity. Images were saved in multipage TIFF format, imported into Amira (Visage Imaging) and manually segmented to identify the cell and the surrounding hydrogel. A 2D surface mesh of the cell was generated from the segmented image, simplified to the desired number of elements and smoothed using built-in functions. This mesh was then imported into Hypermesh (Altair) as a stereolithography file. To approximate an infinite medium, we generated a 400-μm cube centered on the cell, seeded the edges with nine nodes (element size of 50 μm), and generated a 2D quadrilateral surface mesh. Using these two surface meshes as a template, we then generated a 3D tetrahedral mesh (four-node linear tetrahedron elements ‘C3D4’ in Abaqus) of the enclosed volume. These meshes were then imported into Abaqus (Dassault Systémes) for finite element analysis with the bottom surface of the cube fixed as a boundary constraint. Validity of the finite element approximation of an infinite medium was verified by fixing the top surface of the cube as an additional boundary constraint and showed no substantial difference in the recovered tractions. Unless otherwise mentioned, for all measurements, the cells were discretized using 2,000 linear elements. The center of mass of the cell was computed using the area-weighted centroids of each element on the 2D surface mesh of the cell. Renderings of cellular tractions were computed in Tecplot 360 (Tecplot Inc.), and contour plots were scaled such that ˜1% of all elements on the cell were saturated. The deviation of the tractions fields from static equilibrium was assessed by summing the projection of the forces (tractions multiplied by facet area) on each facet of the cell along each Cartesian direction. All data presented in the manuscript were calculated using a Dell Precision T7400 workstation equipped with dual quad core Intel Xeon processors and 16 GB of RAM.

### Mechanical Characterization of Hydrogel Substrates.

The shear modulus of swollen gels was obtained using an AR 2000 oscillating rheometer (TA Instruments) on a temperature-controlled Peltier plate at 37° C. and a 20-mm stainless steel plate with solvent trap geometry (TA Instruments). Cylindrical gel samples were created from 125 μl of identical precursor solution to that used for traction measurements, covalently linked to glass microscope slides treated with 3-(trimethoxysilyl)propyl methacrylate (Sigma) and then swollen in growth medium at 37° C. and 5% CO₂ for 72 h Immediately before testing, the slides were removed from medium and carefully blotted dry with laboratory wipes. The heights and diameter of the swollen gels were measured with calipers and were typically ˜0.5-mm thick and had a ˜19-mm diameter. To prevent slipping, 400 grit, wet-dry sandpaper was sectioned to fully cover the geometry and attached with double-stick tape. The head was lowered to a gap corresponding to approximately 0.2 N of normal force. Three consecutive controlled oscillatory strain sweeps were performed from 0.1-50% radial strain with 30 linearly spaced measurements at 0.25 Hz. After the strain sweeps, frequency sweeps were performed from 0.1-10 Hz, ten measurements per decade on a log scale, at 1% controlled strain. These data were acquired for six independent samples from multiple experiments. The data from the strain sweeps were averaged to yield a shear modulus of 196±66 Pa, 328±76 Pa and 267±34 Pa (±s.d.) for 10% (w/v) PEGDA, 11% (w/v) PEGDA and 7% (w/w) PEGDAam hydrogels, respectively. These values were used to calculate Young's moduli of 585±196 Pa, 978±228 Pa and 796±102 Pa (±s.d.; assuming a Poisson's ratio of 0.49).

To characterize the validity of a homogeneous material assumption, cell-laden degradable matrices were prepared as described above, cultured for 72 h, labeled with Cell Tracker Red (Invitrogen) according to the manufacturer's instructions and then treated with 0.5% SDS. Nondegradable matrices were prepared in an identical manner using PEGDA (MW, 6000; Sigma) in absence of degradable peptides and measured after 48 h. These matrices were imaged before and after applying a uniform compression of approximately 5% strain using a microscope mounted micromanipulator pressed against a coverslip laid over the gel, and bead displacements throughout the volume were computed between the unstressed and compressed images. A 3D tetrahedral mesh was constructed in the vicinity of a cell as described above, and nodal displacements of the boundary nodes were interpolated from the experimentally observed bead displacements. The forward finite element solution was then solved for static equilibrium under homogeneous or heterogeneous (that is, weakening near the cell) material assumptions, and predicted bead displacements in the simulated volume were compared to experimental observations.

### Measurement of Uncertainties in the Displacement Field and Discretized Cell Surface, and Validation Using Simulated Data.

The errors of the displacement measurements were measured from bead displacements before and after treatment with 0.5% SDS in six separate regions of the gel that contained no cells from multiple experiments. These six datasets were used to accurately reflect our experimental bead distribution and displacement resolution in all numerical simulations. To determine the uncertainty in our discretization of the cell surface, two separate surfaces were generated (starting with raw confocal data, proceeding through manual image segmentation and finally to surface reconstruction) of seven cells from multiple experiments. The differences between the two surface meshes for each cell were computed by finding the minimal distance between the nodes of one surface and the closest plane of the alternate surface. To model the cell in our numerical analysis, we used a simplified geometry of a 50-μm-diameter sphere meshed using 2,000 triangular elements and generated a 3D tetrahedral mesh as described above. We first tested our ability to recover a uniform traction of 183 Pa oriented in the outward normal direction on each facet. The forward solution for this loading was solved, and bead displacements were measured at the centroid locations of each bead for each of the six fields measured above, thus giving six separate datasets of simulated bead displacements. The tractions were recovered for each of these simulated displacement fields and compared to the applied loading, thus giving a measurement of the mean error and deviation of the recovered tractions. To simulate the effect of bead displacement noise on the recovered tractions, the experimentally measured displacements from each of the six noise fields were superimposed on the displacement resulting from the simulated loadings, and the tractions were recomputed. To simulate the effect of surface discretization error, for each node of our spherical surface mesh, we randomly sampled measurements of the surface discretization error (computed as described above). As the most accurate discretization can be expected to lie in between the two experimentally generated surfaces, the spatial coordinates of each node from our spherical mesh were shifted either in the inward or outward normal direction (chosen randomly) by one half the magnitude of the experimentally measured noise. The restriction of the noise to the normal directions was necessary to avoid element intersections. This procedure was repeated to generate six independent samples of the surface-discretization noise (that is, we generated six independent ‘noisy’ spherical surfaces) on which to recover tractions.

To test the spatial resolution of the recovery, we applied oscillatory loadings normal to the cell surface. The magnitudes of these loadings varied sinusoidally with respect to the spherical coordinate θ. Three loadings were chosen with peak amplitudes of ±183, ±743 and ±1467 Pa. The frequency of these loadings was then increased progressively from two to ten oscillations per 360°, and six separate measurements of the recovered tractions were obtained for each loading. The characteristic length of the simulated loadings was calculated as the average period of oscillation on the surface of the sphere. The relative error between the simulated and recovered loadings was computed by summing over all elements as:

Relative Error=|T_{recovered}−T_{simulated}|²/|T_{simulated}|²

where Trecovered and Tsimulated are n×3 matrices containing the recovered and simulated tractions respectively, n is the number of facets used to discretize the cell and each row contains the Cartesian components of the traction computed at a given facet. In this manner, a value of 0 indicates perfectly recovered tractions, 1 indicates that the errors are of equal magnitude to the simulated loadings and a value of greater than 1 indicates that the errors are larger than the simulated loadings. For cases in which this value was 0-1, it was possible to express a percentage traction recovery as (1−relative error)×100.

### Cell Culture.

NIH 3T3 cells (American Type Culture Collection; ATCC) were maintained in high-glucose DMEM containing 10% bovine serum, 2 mM L-glutamine, 100 units ml-1 penicillin and 100 mg ml-1 streptomycin (all from Invitrogen). Cell culture medium was changed every 3 d. EGFP-lentiviral infection was carried out as described previously19. Human mesenchymal stem cells from Lonza and Lewis lung carcinoma (LLC) cells from ATCC were maintained in growth medium (low-glucose DMEM containing 10% FBS, 0.3 mg ml⁻¹ glutamine, 100 units ml⁻¹ streptomycin and 100 units ml⁻¹ penicillin). Immediately upon encapsulation of single LLC cells, the medium was supplemented with 50 ng ml⁻¹ of recombinant human hepatocyte growth factor (R&D systems) to drive proliferation and spheroid formation.

What is claimed:

1. A macromer comprising at least one unit of the formula
P-(protein-P)ₙ
wherein:
P is selected from polyethylene glycol (PEG), alginate, polyurethane, and polyvinyl alcohol;
protein comprises at least one bis-cysteine matrix metalloproteinase (MMP)-sensitive peptide or bis-amine protein; and
n is an integer from 2 to 500;
said macromer having a molecular weight of at least 2 kDa.

2. The macromer of claim 1, wherein P is PEG and n is an integer that is in the range of 50 to 150.

3. The macromer of claim 1, wherein P is PEG having a molecular weight of about 2,000 to 40,000 Da.

4. The macromer of claim 1, where said protein comprises at least one peptide having the sequence CGPQGIAGQGCR, CGPQGPAGQGCR or CGPQGIWGQGCR.

5. The macromer of claim 1, wherein said protein is a bis-cysteine matrix metalloproteinase (MMP)-sensitive peptide.

6. The macromer of claim 1, wherein said macromer is associated with at least one additional macromer as defined in claim 1, said macromers being associated via one or more of cross-linking, hydrogen bonding or ionic or van der Waals interactions.

7. The macromer of claim 1, wherein said protein additionally comprises non-MMP-sensitive peptides.

8. The macromer of claim 1, wherein P comprises one or more of alginate, polyurethane, and polyvinyl alcohol

9. The macromer of claim 1, wherein said protein comprises an enzyme.

10. The macromer of claim 1, wherein said protein comprises a biologic growth factor.

11. A hydrogel tissue engineering scaffold comprising a hydrogel derived from cross-linking of a macromer of claim 1.

12. The hydrogel tissue engineering scaffold of claim 11, wherein P is PEG having a molecular weight of about 2,000 to 40,000 and n is an integer that is in the range of 50 to 150.

13. The hydrogel tissue engineering scaffold of claim 11, where said protein comprises at least one peptide having the sequence CGRGDS, CGRGES, CGPQGIAGQGCR, CGPQGPAGQGCR or CGPQGIWGQGCR.

14. The hydrogel tissue engineering scaffold of claim 11, wherein said PEG is substantially linear.

15. A method of producing a bioactive hydrogel comprising:
step-growth polymerization of (i) protein comprising one or more bis-cysteine matrix metalloproteinase (MMP)-sensitive peptides and (ii) at least one of polyethylene glycol-divinylsulfone, polyethylene glycol-diacrylate, polyethylene glycol-diacrylamide and PEG-dicarboxylic acid or derivatives thereof, to produce macromers of the formula (X-PEG-(peptide-PEG)n-X, at least 50% of said macromers having a molecular weight of at least 2 kDa; and
cross-linking said macromers to form said bioactive hydrogel;
wherein X is carboxylic acid, vinylsulfone, acrylate or acrylamide, PEG is polyethylene glycol, and n is 2 to 500.

16. The method of claim 15 wherein n is 50 to 150.

17. The method of claim 15, wherein said protein comprises at least one peptide having the sequence CGPQGIAGQGCR, CGPQGPAGQGCR or CGPQGIWGQGCR.

18. The method of claim 15, wherein said bis-cysteine matrix metalloproteinase (MMP)-sensitive peptide comprises at least one of CGPQGIAGQGCR, CGPQGPAGQGCR and CGPQGIWGQGCR.

19. The method of claim 15, wherein said PEG has a molecular weight of 2,000 to 40,000 Da.

20. The method of claim 16, wherein said step-growth polymerization was accomplished by Michael-type addition in aqueous solution having a basic pH.

21. The method of claim 20, wherein said step-growth polymerization occurs with a molar excess of (i) the moles of polyethylene glycol-diacrylate or polyethylene glycol-diacrylamide relative to (ii) the moles of bis-cysteine matrix metalloproteinase (MMP)-sensitive peptide.

22. The method of claim 15, wherein said cross-linking is accomplished by radical mediated photopolymerization.

23. The method of claim 15, wherein said cross-linking is accomplished by hydrogen bonding or ionic interactions between said protein segments.

24. The method of claim 15, wherein said step-growth polymerization to form the macromer is accomplished in an organic solvent.

25. The method of claim 15, wherein at least 90% of said macromers have a molecular weight of at least 500 kDa.

26. The method of claim 15, wherein the PEG-diacrylate or PEG-diacrylamide are instead PEG-divinylsulfone and X is therefore vinylsulfone.

27. The method of claim 15, wherein the PEG-dicarboxylic acid or derivatives thereof, is PEG-di-N-hydroxysuccinimide or PEG-di-succinimidylcarboxymethylester.

28. The method of claim 15, wherein a mixture of acrylate-PEG-N-hydroxysuccinimide or acrylamide-PEG-N-hydroxysuccinimide and PEG-di-N-hydroxysuccinimide is used in the step-growth polymerization step.

29. The method of claim 15, wherein said step-growth polymerization to form the macromer is accomplished with ‘living’ polymerization methods between polyethylene glycol-diacrylate and polyethylene glycol-diacrylamide chains and bis-acrylate flanked amino acid sequences previously listed including metal ion catalyzed anionic and cationic polymerization.

30. The method of claim 30, wherein said step-growth polymerization to form the macromer is accomplished with ‘living’ radical polymerization methods including reversible addition-fragmentation chain transfer (RAFT) using reversible transfer agents and transition metal catalyzed atom transfer radical polymerization (ATRP).

31. The method of claim 31, wherein said step-growth polymerization is controlled and defined a priori with block copolymer arrangements as dictated by order of reagent addition in polymerization.

32. The method of claim 31, wherein said step-growth polymerization is controlled to narrow polydispersity (<1.2).

33. The method of claim 15, wherein said step-growth polymerization to form the macromer is accomplished with radical thiol-ene ‘click’ reaction with appropriate radical imitator.

34. The method of claim 33, wherein said step-growth polymerization is designed to occur between multifunctional monomers capable of generating thiol-acrylate reactions and, in addition, to orthogonal functionalities present on the monomers for further functionalization using additional ‘click’ chemistries.


SEQUENCE LISTING (9 sequences)

SEQ ID NO: 1 - protein, 6 aa, Artificial Sequence
  Description of Artificial Sequence Synthetic peptide
CGRGDS

SEQ ID NO: 2 - protein, 6 aa, Artificial Sequence
  Description of Artificial Sequence Synthetic peptide
CGRGES

SEQ ID NO: 3 - protein, 12 aa, Artificial Sequence
  Description of Artificial Sequence Synthetic peptide
CGPQGIAGQGCR

SEQ ID NO: 4 - protein, 12 aa, Artificial Sequence
  Description of Artificial Sequence Synthetic peptide
CGPQGPAGQGCR

SEQ ID NO: 5 - protein, 12 aa, Artificial Sequence
  Description of Artificial Sequence Synthetic peptide
CGPQGIWGQGCR

SEQ ID NO: 6 - protein, 6 aa, Artificial Sequence
  Description of Artificial Sequence Synthetic peptide
CGREDV

SEQ ID NO: 7 - protein, 4 aa, Artificial Sequence
  Description of Artificial Sequence Synthetic peptide
RGDS

SEQ ID NO: 8 - protein, 4 aa, Artificial Sequence
  Description of Artificial Sequence Synthetic peptide
RGES

SEQ ID NO: 9 - protein, 9 aa, Artificial Sequence
  Description of Artificial Sequence Synthetic peptide
GPQGIWGQK